(19) United States
(12) Patent Application Publication
(10) Pub. No.: US 2005/0202065 A1
(43) Pub. Date: Sep. 15, 2005

(54) Orange juice of low citrus oil content for reduction of heartburn episodes

(75) Inventors: Richard N. McArdle, Bradenton, FL (US); Stephen A. Letourneau, Holmes Beach, FL (US); Albert D. Bolles, Bradenton, FL (US); Carla R. McGill, Sarasota, FL (US); Nancy R. Green, Bradenton, FL (US)

Correspondence Address: RYNDAK & SURI, 30 NORTH LASALLE STREET, SUITE 2630, CHICAGO, IL, 60602, US

(73) Assignee: Tropicana Products, Inc.

(21) Appl. No.: 11/123,949

(22) Filed: May 6, 2005

Related U.S. Application Data
(63) Continuation of application No. 10/376,439, filed on May 7, 2003, now abandoned.
(63) Continuation-in-part of application No. 09/848,675, filed on May 3, 2001, now Pat. No. 6,682,767.

(51) Int. Cl. A61K 35/78

(52) U.S. Cl. 424/439; 424/736

(57) ABSTRACT

Reducing heartburn episodes is achieved in individuals having an orange juice intolerance or food allergy. The orange juice product has a low oil condition, which optionally can be combined with addition of a calcium source. Individuals prone to orange juice induced heartburn episodes experience a reduced incidence of heartburn episodes when they ingest the juice.

### CROSS REFERENCE TO RELATED APPLICATION

This a continuation-in-part of application Ser. No. 09/848,675, filed May 3, 2001, incorporated by reference hereinto.

### BACKGROUND OF THE INVENTION

### Field of the Invention

This invention relates to approaches for reducing heartburn episodes when an individual having an orange juice intolerance ingests orange juice according to the invention. The invention is achieved without any substantial negative impact on orange juice flavor or other important attributes. The orange juice has a low oil condition and also can incorporate a calcium source.

Numerous individuals have been known to experience negative effects upon ingesting different foods. A true food allergy occurs when the immune system of the individual overreacts to certain proteins in food. It is believed that hundreds of food ingredients can provoke an allergic reaction. Typical foods in this regard are nuts, peanuts, milk, eggs, fish, shellfish, soybeans and wheat. Foods such as these can lead to symptoms including nausea, hives, skin rash, nasal congestion, wheezing, and the like. However, most unpleasant reactions to food are caused not by allergies but by intolerances, which tend to be less severe than true food allergies. Typical in this regard are lactose intolerance, sulfite intolerance and intolerance to monosodium glutamate, red wine, chocolate and food coloring agents. Another intolerance of some frequency is manifested by gastral distress and/or digestive difficulties which certain individuals experience shortly upon ingesting orange juice products.

In some circles, it is generally assumed that the relatively high acidity of orange juice products is a primary contributor to these negative or unpleasant experiences with orange juice products for a small percentage of the population. For example, Kligerman et al U.S. Pat. No. 5,665,415 and U.S. Pat. No. 5,869,119, incorporated hereinto by reference, suggest that acidic foods or beverages such as coffee and other beverages can be combined with calcium glycerophosphate so as to raise the pH of the food or beverage by at least 0.5 pH units, such as to a pH of greater than 5.4, which typically is a pH higher than desirable for superior tasting orange juice. This pH adjustment is said to reduce the tendency of the food or beverage to cause heartburn and other esophageal and/or gastrointestinal distress. This approach generally follows the conventional wisdom that ingesting antacids treats heartburn by helping to neutralize stomach acid. This approach suggests, in general, raising the pH of the food or beverage to well above 5.

Other approaches have suggested acid reduction for relieving symptoms such as the burning, painful sensation of heartburn. Included is Georgiades et al. U.S. Pat. No. 5,762,962, incorporated hereinto by reference. This patent is directed to antacid pharmaceutical compositions comprising a combination of calcium salts. Another pharmaceutical is found in Korn et al. U.S. Pat. No. 5,989,588 which suggests administering to a patient for preventing heartburn a composition having a pharmaceutically effective amount of an H2 antagonist such as famotidine. These antacid approaches administer tablets in a manner customary for over-the-counter or pharmaceutical antacid administration.

In addition it is well-known that beverages such as orange juice can be supplemented with calcium with the objective of addressing inadequate calcium in the diets of individuals. Numerous approaches have been proposed or implemented in this regard. Included is the technology in patents such as Meyer et al U.S. Pat. No. 5,474,793, Camden et al U.S. Pat. No. 5,225,221, and Heckert U.S. Pat. No. 4,722,847, each incorporated hereinto by reference. These take the approach of adding to fruit juices a source of calcium, at times together with a mixture of citric acid and malic acid.

Other approaches suggest the use of calcium for drink fortification. Keating U.S. Pat. No. 5,500,232 and U.S. Pat. No. 5,834,045, incorporated hereinto by reference, describe shelf-stable beverages to which are added an acidulant and a source of calcium having calcium hydroxide and calcium glycerophosphate.

These various approaches do not directly address the problem faced by individuals who wish to alleviate discomfort generally falling under the category of heartburn and which can be associated with drinking orange juice. Previous approaches focus on reducing acidity, either within the juice itself, or by administering antacid tablets in an effort to combat acidity within the digestive tract of the individual drinking the orange juice. Approaches heretofore have not satisfactorily arrived at orange juice products themselves which directly address the incidence of heartburn episodes in those individuals who have an orange juice intolerance, insensitivity or allergy. There accordingly is a need for an approach which is more effective than acidity reduction and that is more convenient and self-contained than is the antacid tablet approach.

### SUMMARY OF THE INVENTION

In accordance with the present invention, orange juice is provided which reduces the incidence of heartburn episodes in individuals having orange juice intolerance. An orange juice supply is modified and/or provided to impart a lower oil condition to the orange juice product so that the concentration of citrus oil in the orange juice product is less than 0.01 volume percent, according to the Scott oil method. Typically, the Scott oil method detects and measures the effects of compounds which add bromine across double bonds, and is an industry standard for measuring oil content in citrus juices.

Heartburn episodes are reduced for an individual having orange juice intolerance when compared with the incidence of heartburn episodes by that same individual ingesting the orange juice supply which does not include these characteristics. The overall negative impact of any oil remaining in the juice optionally can be addressed by including a calcium source so that the calcium concentration within the orange juice product is greater than 0.04 weight percent, based upon the total weight of the orange juice product.

A general object of the present invention is to provide a method and product for reducing the incidence of heartburn episodes in an individual having an orange juice intolerance.

Another object of the present invention is to provide an approach for reducing heartburn episodes by avoiding the episodes, rather than by administering a treatment agent to the individual, by providing an orange juice product which does not antagonize the individual so that heartburn symptoms develop.

Another object of this invention is to provide an improved method and product for safe ingestion of orange juice without experiencing gastrointestinal discomfort.

Another object of the present invention is to provide a modified orange juice supply having low citrus oil, which produces a situation by which heartburn discomfort by orange juice ingestion is lessened.

Other objects and advantages of the present invention will be understood from the following description according to preferred embodiments of the present invention, relevant information concerning which is shown in the accompanying drawings.

### BRIEF DESCRIPTION OF THE DRAWINGS

FIG. 1 is a plot of data of heartburn rating over time collected during evaluation of certain From Concentrate (FC) orange juice products.

### DESCRIPTION OF THE PREFERRED EMBODIMENTS

Orange juice products are provided which have been found to alleviate gastrointestinal distress in individuals which tend to have negative or uncomfortable gastrointestinal experiences upon ingesting orange juice. Such individuals experience post-ingestive symptoms of so-called acid reflux or heartburn. Included in the orange juice products, which are included as an aspect of the invention, is an orange juice supply having low oil characteristics.

According to this invention, the citrus oil content of an orange juice product is a primary contributor the distress experienced by those individuals having an orange juice intolerance or food allergy. Low oil characteristics refer to the concentration of citrus oil within the juice product. Citrus oil is generally understood in the art as being the component of citrus juice at a concentration measured by the Scott oil method. As noted earlier, the Scott oil method is well known in the citrus juice art for measuring oil content as a volume measurement. A typical citrus oil content is primarily a terpene content which originates to a large extent from citrus peel. A typical major terpene in this regard is d-limonene. This is by far the primary terpene in orange juice.

Citrus oil content within the orange juice product can range from virtually zero percent oil to not greater than about 0.01 volume percent oil, based upon the total weight of the oil juice product. As used herein “low oil” or “very low oil” can encompass products which have no detectible oil levels, which could be called “no oil” products, as well as products having maximum oil levels which are specified herein.

Generally, in accordance with the invention the lower the citrus oil content, the more likely that the reduction of heartburn episodes will be realized. Preferred citrus oil range upper limits for not from concentrate (NFC) orange juices are not greater than about 0.01 percent oil, more preferably not greater than about 0.005 percent oil, most preferably not greater than about 0.003 percent oil. A preferred very low oil concentration, typically for from concentrate (FC) orange juices is not greater than about 0.005 percent oil, more preferably not greater than about 0.003 percent oil, most preferably not greater than about 0.001 percent oil. These percentages are determined according to the Scott oil method, which is a volume per volume measurement. A citrus oil concentration above some of these indicated preferred upper citrus oil levels can experience heartburn episodes reduction when combined with the calcium source addition aspect of the invention, when desired.

A typical low-oil condition can include consideration of terpene levels. Usually, terpene levels would be at a terpene concentration of less than about 80 ppm of terpene in the juice product. Preferably, the terpene level is less than about 60 ppm terpene in the juice product. Most preferably, the terpene content is less than about 40 ppm terpenes in the juice product. An especially low oil condition corresponds to a terpene concentration of less than about 20 ppm terpene in the juice product.

Referring further to adding a calcium source to the low oil orange juice supply, when practiced this step provides a calcium concentration within the orange juice that is greater than 0.04 weight percent, based upon the total weight of the orange juice. The Ca levels are measured by a standard wet chemistry analysis of titration, such as with ethylene diaminetetracetic acid. While not wishing to be bound by any particular mechanism for achieving the objects herein, it is believed that the calcium source addition has at least two advantageous effects.

One effect is that a calcium source lowers somewhat the titratable acidity. This can be considered as a mechanism which achieves a lowered titratable acidity which is selected for the product. While the concept of titratable acidity is well-known to those in the art, the preferred test for measuring titratable acidity is the standard method of titration of citric acid with sodium hydroxide.

Another mechanism which is believed to occur with the addition of a calcium source can be loosely characterized as assisting in negating the distress-aggravating effects of the citrus oil content of the juice supply. This is believed to be particularly evident when the citrus oil content of the orange juice supply being used is higher than a preferred citrus oil content or is somewhat on the high side of one of the citrus oil content ranges in order to achieve the advantageous results of the invention.

When the calcium source is a calcium citrate source, typically same is provided as a powdered tetrahydrate. Calcium citrate preferably is provided as tricalcium citrate tetrahydrate. Other calcium sources include calcium malate, calcium hydroxide, calcium glycerophosphate, and food acid salts of calcium, inculding calcium phosphate, calcium lactate and calcium gluconate, and various other calcium sources as suitable for the particular low oil juice. It will be appreciated that most orange juice sources already have a relatively low quantity of calcium. The concentration of calcium is increased by the calcium source addition such that the concentration of calcium within the orange juice product is greater than 0.04 weight percent and equal to or less than 0.2 weight percent. It will be appreciated that calcium levels within higher portions of this range can produce juice products which are characterized as containing a calcium supplement. It is not required that the calcium source addition be to a level at or in excess of that which a claim for a calcium supplemented juice can be made. Rather, it is contemplated that calcium levels can be at lower levels. Thus, the advantages of the calcium source addition according to this invention typically are achieved without any need to be as high as that required to provide a juice which can be labeled as a calcium supplemented orange juice.

In fact, it is generally preferred that the calcium source addition be at a level below that at which taste or other sensory parameters will be affected negatively or will otherwise deviate from a chosen norm. Levels above about 0.065 weight percent calcium in the orange juice product tend to exhibit these effects. Accordingly, when these effects are to be avoided, the calcium content should be below about 0.07 weight percent, more comfortably below about 0.06 weight percent. An especially preferred calcium content range for products according to the invention for many juice sources is between above 0.04 weight percent and below 0.05 weight percent.

Addition of the calcium source into the orange juice supply can be carried out by any suitable means of adding a calcium source and which is appropriate for industrial-scale operations. Typically, a conventional dry solids mixing system is adequate for a powdered or particulate product. Other systems are known and available for the addition of a liquid type of calcium additive.

A further aspect which can be considered in connection with reduction of heartburn episodes involves the solids content of the juice. A so-called low solids orange juice product is prepared by centrifuging a base of orange juice concentrate and water within a centrifuge in order to remove bottom solids so as to provide an analysis of zero solids. This can be accompanied by the addition of citrus oil to a level substantially lower than a natural citrus oil level, but not necessarily within the lower portions of the citrus oil ranges specified herein.

Referring particularly to the method for reducing the incidence of heartburn episodes or other distress experienced by individuals having an orange juice intolerance or allergy, an orange juice supply first is provided. This supply is modified by imparting the low oil condition to the orange juice supply, at times with a calcium source addition as well. A typical orange juice product thus prepared has a pH between about 3.7 and about 4.4.

The result is an orange juice product having characteristics which safeguard and/or insulate the individual from heartburn causation so as to reduce the incidence of heartburn episodes. More particularly, the method achieves a reduction in the incidence of heartburn episodes in an individual having difficulties with orange juice ingestion, this reduction being when compared with the incidence of heartburn episodes by that individual ingesting an orange juice supply which does not have the characteristics of the juice described herein.

This method achieves these effects without significantly detrimentally affecting the sensory attributes of the citrus juice. These sensory attributes include taste and especially mouthfeel of the juice. In most instances, the juice products carrying out the method exhibit a mouthfeel and/or taste which is recognized as being smoother than orange juice not having the characteristics disclosed herein.

Studies were undertaken in order to evaluate heartburn episode reduction as reported in the following Examples.

### EXAMPLE 1

Three From Concentrate orange juice products were formulated as follows. In these, all weight percents were rounded to 0.01%, and they are based upon the total weight of the orange juice product, unless otherwise specified.

A Control FC orange juice was prepared by combining 17.64 weight percent orange juice concentrate (65° brix) with 82.33 weight percent water and 0.03 volume percent added citrus oil. The Control FC orange juice product was a conventional product produced by diluting no-oil added orange juice concentrate with water (to 12.2° brix) and adding orange oil to a concentration of 0.025 volume percent. This Control was heat pasteurized in accordance with usual industry practices. All of the products of this Example were pasteurized and held refrigerated in bottles until use.

A Low Oil No Solids orange juice was prepared by combining 17.64 weight percent of the concentrate with 82.33 weight percent of water, and this was centrifuged to remove bottom solids until a 0 solids analysis was achieved. Thereafter, citrus oil was added at a level of 0.03 volume percent. The oil concentration in the low oil no solids juice was 0.016 volume percent according to the Scott oil method.

FC orange juice having a very low oil value was prepared. It was labeled Very Low Oil orange juice. This product was made by providing 17.64 weight percent of the same concentrate, 82.35 weight percent water, and 0.004 volume percent of a low citrus oil, which had a very low terpene content (primarily d-limonene) according to a standard Scott oil analysis. This Very Low Oil orange juice was created by diluting the same pumpout concentrate used in making the other products (including the Control) with water and adding orange oil components having very low terpene levels. This produced an orange juice having a profile similar to that of the Control, but having extremely low oil analysis (only about 12% of that of the Control). The oil concentration was 0.003 volume percent according to the Scott oil method.

Healthy male and female adult volunteer subjects were screened for their ability to perceive digestive difficulties with orange juice. Each recruit participated in four screening sessions. After an overnight fast, each subject was provided with 8 ounces of either orange juice or a placebo beverage (apple juice) in a styrofoam cup having an opaque lid and straw to obscure visual difference. Each subject was requested to rate post-ingestive symptoms over one hour at 15 minute intervals. On three of the four screening occasions, the screening beverage was regular orange juice, and on the other occasion, the placebo was the beverage ingested. Individuals who reported symptoms for two of the three orange juice trials and had no reaction to the placebo were admitted to the study. The study had 20 subjects.

The three juice products of this Example were evaluated in duplicate during eight sessions in random order, the sessions being separated by at least one day. The qualified subjects recorded their reactions (self-perceived) to each product when tested by placing a mark on a generic human figure representing the area of discomfort for that subject during that test event. At that time, each subject gave the designated symptom a numerical rating. Ratings were recorded when the subjects first ingested each sample and at 15, 30, 45, and 60 minute intervals.

The relevant chemical parameters and heartburn response ratings at one hour after ingestion of each type of product are reported in Table I. These responses are reported as Mean Response values.

TABLE I

| | | Low Oil | |
|---|---|---|---|
| | Control | No Solids | Very Low Oil |
| Brix | 12.2 | 12.2 | 12.2 |
| Acid, % | 0.66 | 0.66 | 0.67 |
| Ratio | 18.41 | 18.55 | 18.15 |
| PH | 3.90 | 4.01 | 3.88 |
| Bottom Solids | 10.0 | 0.0 | 10.0 |
| Oil, % | 0.025 | 0.016 | 0.003 |
| Calcium, | 9.0 | 8.9 | 9.1 |
| mg/100 mL | | | |
| Vitamin C, | 49.3 | 41.6 | 50.0 |
| mg./100 mL | | | |
| Limonene, ppm | 113 | 67 | 7.6 |
| Mean Response | 1.6a | 1.5ab | 1.4b |

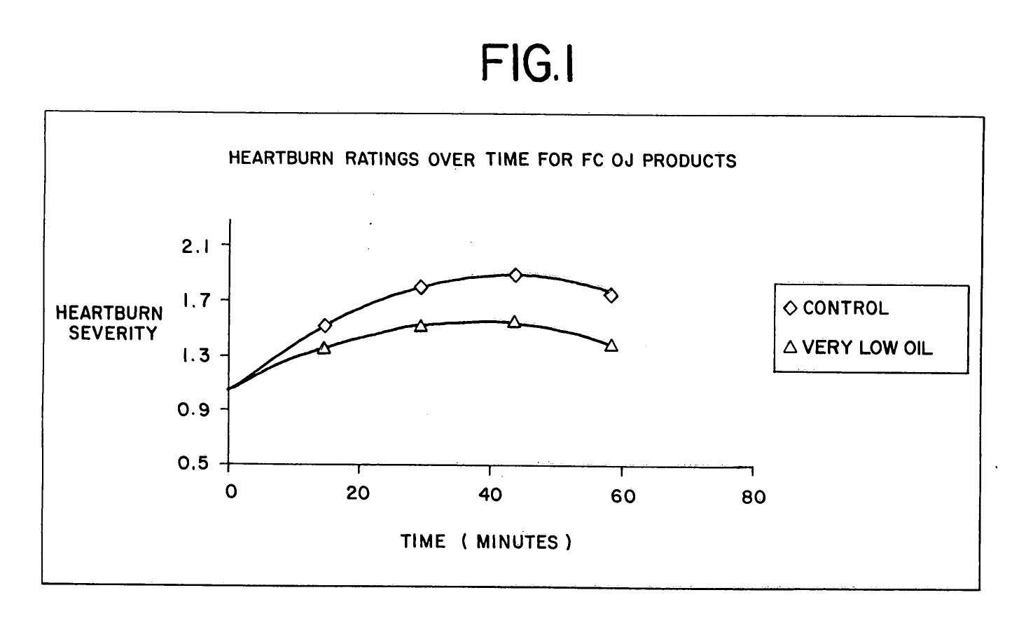

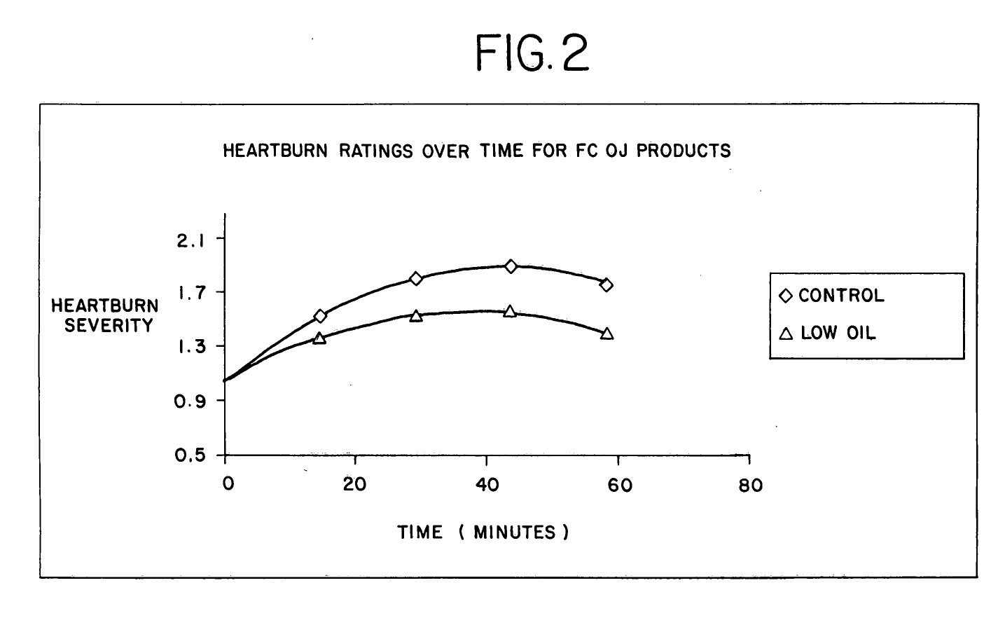

The Mean Response values reported in Table I are at one hour after ingestion. Means having the same letter are not significantly different at α=0.01. A standard LSD analysis was used. These Mean Responses indicate that the Very Low Oil product reported significantly reduced heartburn symptoms relative to the Control product, strongly indicating a relationship between citrus oil and/or terpene and heartburn symptoms. Although the solids were removed in the No Solids product, about half of the limonine and more than half of the oil remained from the Control, and the full beneficial effect of the Very Low Oil FC product was not realized. FIG. 1 provides an indication of the consistency of the heartburn severity reduction over the rating time intervals for the Very Low Oil product versus the Control product.

### EXAMPLE 2

Four male and female subjects volunteered to evaluate orange juice products. Each subject indicated he or she had a history of digestive problems with orange juice. Five products were tested. Each product began with the same so-called “pump out” concentrate, a concept well known in the art. The particular pump-out concentrate used a very low terpene content according to Scott oil analysis.

A Control product was made from 17.64 weight percent of the pump out concentrate (65° brix), 82.33 weight percent water, and 0.03 volume percent citrus oil. Weight percents in this Example were rounded to 0.001 percent. The Control product had a low-acid characteristic (0.5 weight percent titratable acidity), and each of the other products were made from this Control pump-out concentrate and had the same or similar titratable acidity values. This Control had a typical FC oil level of 0.025 volume percent and a typical limonene content of 150 ppm (0.015 weight percent).

Two of these FC orange juice products were prepared to have no citrus flavor oil, and each had non-detectable levels of terpenes. One of these was identified as the No oii orange juice of this Example, having a titratable acidity of 0.5 weight percent and a calcium content of 13 mg Ca per 100 ml of juice product (about 0.012 weight percent). This No Oil product was comprised of 17.64 weight percent of the concentrate of the Control and 82.36 weight percent water.

The other product having non-detectable levels of terpenes was identified as No Oil+Calcium. Calcium glycerophosphate was added. This No Oil+Calcium orange juice product had a titratable acidity of 0.51 weight percent, a zero oil content, and 150 mg Ca per 100 ml of juice product (about 0.14 weight percent calcium). This No Oil+Calcium product was prepared from 17.5 weight percent of the concentrate, 81.68 weight percent water, and 0.82 weight percent calcium glycerophosphate.

Another product was prepared with this same level of added calcium glycerophosphate and had a moderate oil level noticeably lower than that of the control, namely 0.018 volume percent. Its titratable acidity was 0.51 weight percent. This was identified as the Low Oil+Calcium product. The Low Oil+Calcium product was prepared from 17.49 weight percent of the concentrate, 81.66 weight percent water, 0.03 volume percent added citric oil, and 0.82 weight percent calcium glycerophosphate.

Also prepared was a Very Low Oil formulation which was virtually identical to the Very Low Oil product of Example 1.

The brix level for each product was targeted at 12.2° brix, although the product with added calcium analyzed at a higher brix level because calcium adds to the brix level for refractive index reasons.

Each subject received 6-ounce samples three days a week over an eight-week period. The samples were given to the subjects in opaque cups with lids and an opaque straw so that the products could not be distinguished visually. Subjects were asked not to eat or drink within two hours of the test. After drinking the juice samples, the subjects rated severity of reaction within two hours. A scale of 1 to 5 was used, with 1 signifying a mild reaction and 5 signifying a severe reaction. Each subject recorded the symptoms he or she observed, as well as the nature of the symptoms. Almost all of the reactions were recorded as heartburn symptoms which could be relieved by water, crackers and/or antacids in most instances.

The chemical analysis for each of the Control and the four products are reported in Table II. In addition, the Mean Response of these heartburn symptoms also is reported in this Table II for each product.

TABLE II

| | | | No Oil + | Low Oil + | Very |
|---|---|---|---|---|---|
| | Control | No Oil | Calcium | Calcium | Low Oil |
| Brix | 12.2 | 12.2 | 12.8 | 12.8 | 12.2 |
| Acid, % | 0.5 | 0.5 | 0.51 | 0.51 | 0.52 |
| Ratio | 24.4 | 24.4 | 25.1 | 25.1 | 23.4 |
| pH | 4.06 | 4.06 | 4.45 | 4.45 | 4.12 |
| Oil, % | 0.025 | 0.0000 | 0.0000 | 0.018 | 0.001 |
| Calcium, | 13 | 13 | 150 | 150 | 13 |
| mg/100 mL | | | | | |
| Limonene, | 150 | 0 | 0 | 56 | 4 |
| ppm | | | | | |
| Mean | 0.9a | 1.0b | 1.1b | 0.7b | 0.9b |
| Response | | | | | |

In this study, each of the non-Control FC products were reported as achieving statistically lower heartburn symptoms than the Control. These data indicate that the relatively low acid content of the Control was not by itself adequate to achieve the heartburn symptom control of the other products of this Example. The mean response indicated improvement in heartburn symptoms by a formulation having a moderate oil content and limonene content, illustrating the effectiveness of Calcium in offsetting negative influences of the oil, even at the somewhat low level of the Low Oil+Calcium product. The No Oil+Calcium product was no more effective than the No Oil product, suggesting redundancy of the added calcium in a formulation with no terpene or other citrus oil.

It will be understood that the embodiments of the present invention which have been described are illustrative of some of the applications of the principles of the present invention. Numerous modifications may be made by those skilled in the art without departing from the true spirit and scope of the invention.

1. A method for reducing the incidence of heartburn episodes in an individual having an orange juice intolerance, comprising:
providing an initial not from concentrate (NFC) orange juice supply;
imparting to said NFC orange juice supply a very low oil condition;
said very low oil condition provides an NFC orange juice having heartburn-safeguarding characteristics, said orange juice having heartburn safeguarding characteristics being an NFC orange juice product having a concentration of citrus oil which is not greater than about 0.01 volume percent according to the Scott oil method; and
ingesting said NFC orange juice product having heartburn-safeguarding characteristics by an individual having an orange juice intolerance such that the incidence of heartburn episodes by that individual is reduced over the incidence of heartburn episodes by said individual ingesting said initial NFC orange juice supply.

2. The method of claim 1, further including adding a calcium source to said orange juice having a very low oil condition in an amount such that the calcium concentration within the orange juice product is greater than 0.04 weight percent, based upon the total weight of the orange juice product.

3. The method of claim 2, wherein, after said adding of calcium, the calcium concentration is not greater than about 0.2 weight percent, based upon the total weight of the orange juice product.

4. The method of claim 1, wherein said concentration of citrus oil is not greater than about 0.005 volume percent according to the Scott oil method.

5. The method of claim 1, wherein said concentration of citrus oil is not greater than about 0.003 volume percent according to the Scott oil method.

6. The method of claim 4, wherein the orange juice product having heartburn-safeguarding characteristics has a titratable acidity of not greater than about 0.7 weight percent, based upon the total weight of the product.

7. The method of claim 2, wherein the calcium source is a calcium citrate source.

8. The method of claim 7, wherein the calcium source is tricalcium citrate tetrahydrate.

9. The method of claim 7, wherein said adding adds the calcium citrate source as dry solid particulates.

10. The method of claim 1, wherein said imparting of the very low oil condition provides a concentration of terpene in said orange juice product which is less than about 60 ppm of the orange juice product.

11. The method of claim 1, wherein said imparting of the very low oil condition provides a concentration of terpene in said orange juice product which is less than about 40 ppm of the orange juice product.

12. The method of claim 1, further including imparting to said orange juice supply a low solids condition by separating bottom solids therefrom.

13. A method for reducing the incidence of heartburn episodes in an individual having an orange juice intolerance, comprising:
providing an initial not from concentrate (NFC) orange juice supply and imparting to said orange juice supply a low oil condition;
adding a calcium source to said orange juice supply thereby increasing the calcium content;
said low oil condition and said calcium content combine to provide an NFC orange juice having heartburn-safeguarding characteristics, said orange juice having heartburn-safeguarding characteristics being an NFC orange juice product having:
(a) a concentration of citrus oil which is less than 0.01 volume percent, according to the Scott oil method, and
(b) a calcium concentration which is greater than 0.04 weight percent, based upon the total weight of the orange juice product; and
ingesting said NFC orange juice product having heartburn-safeguarding characteristics by an individual having an orange juice intolerance such that the incidence of heartburn episodes by that individual is reduced over the incidence of heartburn episodes by said individual ingesting said initial orange juice supply.

14. The method of claim 13, wherein, after said adding of calcium, the calcium concentration-is not greater than about 0.2 weight percent, based upon the total weight of the orange juice product.

15. The method of claim 13, wherein said concentration of citrus oil is not greater than about 0.005 volume percent according to the Scott oil method.

16. The method of claim 13, wherein said concentration of citrus oil is not greater than about 0.003 volume percent according to the Scott oil method.

17. The method of claim 13, wherein the adding of a calcium source adds the calcium source as dry solid particulates into the orange juice supply.

18. The method of claim 13, wherein said imparting of the low oil condition provides a concentration of terpene in said orange juice product which is less than about 80 ppm of the orange juice product.

19. An NFC orange juice product produced in accordance with the method of claim 1, wherein the NFC orange juice product has said heartburn-safeguarding characteristics.

20. An NFC orange juice product produced in accordance with the method of claim 2, wherein the NFC orange juice product has said heartburn-safeguarding characteristics.

21. An NFC orange juice product produced in accordance with the method of claim 13, wherein the NFC orange juice product has said heartburn-safeguarding characteristics.

22. An NFC orange juice product having heartburn-safeguarding characteristics when ingested by an individual having an orange juice intolerance, comprising:
an NFC orange juice product having a very low oil condition;
said very oil condition provides an orange juice having heartburn-safeguard characteristics, said orange juice product having:
(a) a concentration of citrus oil in said orange juice supply which is not greater than 0.005 volume percent, according to the Scott oil method; and
(b) heartburn-safeguarding characteristics when ingested by an individual having an orange juice intolerance such that the incidence of heartburn episodes by that individual is reduced over the incidence of heartburn episodes by said individual ingesting another orange juice product having a concentration of citrus oil which is greater than 0.005 volume percent according to the Scott oil method.

23. The product of claim 22, wherein the orange juice product further includes an added calcium component such that calcium concentration within the orange juice product is greater than 0.04 weight percent, based upon the total weight of the orange juice product.

24. The product of claim 23, wherein said calcium concentration within the orange juice product is not greater than about 0.2 weight percent, based upon the total weight of the orange juice product.

25. The product of claim 22, wherein said very low oil condition comprises a concentration of terpene in said orange juice product which is less than about 40 ppm of the orange juice product.

26. The product of claim 22, wherein said very low oil condition comprises a concentration of terpene in said orange juice product which is less than about 20 ppm of the orange juice product.

27. An NFC orange juice product having heartburn safeguarding characteristics when ingested by an individual having an orange juice intolerance, comprising:
a low oil orange juice product containing an added citrate source, the NFC orange juice product having:
(a) a concentration of citrus oil in said orange juice supply which is less than about 0.01 volume percent according to the Scott oil method;
(b) a calcium component such that the calcium concentration is greater than about 0.04 weight percent, based upon the total weight of the orange juice product; and
(c) heartburn-safeguarding characteristics when ingested by an individual having an orange juice intolerance such that the incidence of heartburn episodes by that individual is reduced over the incidence of heartburn episodes by said individual ingesting another orange juice product having a citrus oil content greater than about 0.01 volume percent according to the Scott oil method, and having no calcium component.

28. The product of claim 27, wherein the calcium concentration is not greater than about 0.2 weight percent, based upon the total weight of the orange juice product.

29. The product of claim 27, wherein the calcium source is a calcium citrate source.

30. The product of claim 27, wherein said low oil condition is one at which terpene in said orange juice product is at a concentration of less than about 60 ppm of the orange juice product.

31. A method for reducing the incidence of heartburn episodes in an individual having an orange juice intolerance, comprising:
providing an initial from concentrate (FC) orange juice supply;
imparting to said FC orange juice supply a very low oil condition;
said very low oil condition provides an FC orange juice having heartburn-safeguarding characteristics, said orange juice having heartburn safeguarding characteristics being an NFC orange juice product having a concentration of citrus oil which is not greater than about 0.005 volume percent according to the Scott oil method; and
ingesting said FC orange juice product having heartburn-safeguarding characteristics by an individual having an orange juice intolerance such that the incidence of heartburn episodes by that individual is reduced over the incidence of heartburn episodes by said individual ingesting said initial FC orange juice supply.

32. The method of claim 31, further including adding a calcium source to said orange juice having a very low oil condition in an amount such that the calcium concentration within the orange juice product is greater than 0.04 weight percent, based upon the total weight of the orange juice product.

33. The method of claim 32, wherein, after said adding of calcium, the calcium concentration is not greater than about 0.2 weight percent, based upon the total weight of the orange juice product.

34. The method of claim 31, wherein said concentration of citrus oil is not greater than about 0.003 volume percent according to the Scott oil method.

35. The method of claim 31, wherein said concentration of citrus oil is not greater than about 0.001 volume percent according to the Scott oil method.

36. The method of claim 34, wherein the orange juice product having heartburn-safeguarding characteristics has a titratable acidity of not greater than about 0.7 weight percent, based upon the total weight of the product.

37. The method of claim 32, wherein the calcium source is a calcium citrate source.

38. The method of claim 37, wherein the calcium source is tricalcium citrate tetrahydrate.

39. The method of claim 37, wherein said adding adds the calcium citrate source as dry solid particulates.

40. The method of claim 31, wherein said imparting of the very low oil condition provides a concentration of terpene in said orange juice product which is less than about 60 ppm of the orange juice product.

41. The method of claim 31, wherein said imparting of the very low oil condition provides a concentration of terpene in said orange juice product which is less than about 40 ppm of the orange juice product.

42. The method of claim 31, further including imparting to said orange juice supply a low solids condition by separating bottom solids therefrom.

43. A method for reducing the incidence of heartburn episodes in an individual having an orange juice intolerance, comprising:
providing an initial from concentrate (FC) orange juice supply and imparting to said orange juice supply a low oil condition;
adding a calcium source to said orange juice supply thereby increasing the calcium content;
said low oil condition and said calcium content combine to provide an orange juice having heartburn-safeguarding characteristics, said orange juice having heartburn-safeguarding characteristics being an FC orange juice product having:
(a) a concentration of citrus oil which is less than 0.005 volume percent, according to the Scott oil method, and
(b) a calcium concentration which is greater than 0.04 weight percent, based upon the total weight of the orange juice product; and
ingesting said FC orange juice product having heartburn-safeguarding characteristics by an individual having an orange juice intolerance such that the incidence of heartburn episodes by that individual is reduced over the incidence of heartburn episodes by said individual ingesting said initial orange juice supply.

44. The method of claim 43, wherein, after said adding, the calcium concentration is not greater than about 0.2 weight percent, based upon the total weight of the orange juice product.

45. The method of claim 43, wherein said concentration of citrus oil is not greater than about 0.003 volume percent according to the Scott oil method.

46. The method of claim 43, wherein the calcium source is a calcium citrate source.

47. The method of claim 43, wherein the adding of a calcium source adds the calcium source as dry solid particulates into the orange juice supply.

48. The method of claim 43, wherein said imparting of the low oil condition provides a concentration of terpene in said orange juice product which is less than about 40 ppm of the orange juice product.

49. An FC orange juice product produced in accordance with the method of claim 31, wherein the FC orange juice product has said heartburn-safeguarding characteristics.

50. An FC orange juice product produced in accordance with the method of claim 32, wherein the NFC orange juice product has said heartburn-safeguarding characteristics.

51. An FC orange juice product produced in accordance with the method of claim 43, wherein the FC orange juice product has said heartburn-safeguarding characteristics.

52. An FC orange juice product having heartburn-safeguarding characteristics when ingested by an individual having an orange juice intolerance, comprising:
an orange juice product having a very low oil condition;
said very low oil condition provides an FC orange juice having heartburn-safeguard characteristics, said orange juice product having:
(a) a concentration of citrus oil in said orange juice supply which is not greater than 0.003 volume percent, according to the Scott oil method; and
(b) heartburn-safeguarding characteristics when ingested by an individual having an orange juice intolerance such that the incidence of heartburn episodes by that individual is reduced over the incidence of heartburn episodes by said individual ingesting another orange juice product having a concentration of citrus oil which is greater than 0.003 volume percent according to the Scott oil method.

53. The product of claim 52, wherein the orange juice product further includes an added calcium component such that calcium concentration within the orange juice product is greater than 0.04 weight percent, and not greater than about 0.2 weight percent, based upon the total weight of the orange juice product.

54. The product of claim 52, wherein said very low oil condition comprises a concentration of terpene in said orange juice product which is less than about 60 ppm of the orange juice product.

55. The product of claim 52, wherein said very low oil condition comprises a concentration of terpene in said orange juice product which is less than about 40 ppm of the orange juice product.

56. The product of claim 52, wherein said very low oil condition comprises a concentration of terpene in said orange juice product which is less than about 20 ppm of the orange juice product.

57. An FC orange juice product having heartburn safeguarding characteristics when ingested by an individual having an orange juice intolerance, comprising:
a low oil orange juice product containing a calcium citrate source, the FC orange juice product having:
(a) a concentration of citrus oil in said orange juice supply which is less than about 0.005 volume percent according to the Scott oil method;
(b) a calcium component such that the calcium concentration is greater than about 0.04 weight percent, based upon the total weight of the orange juice product; and
(c) heartburn-safeguarding characteristics when ingested by an individual having an orange juice intolerance such that the incidence of heartburn episodes by that individual is reduced over the incidence of heartburn episodes by said individual ingesting another orange juice product having a citrus oil content greater than about 0.005 volume percent according to the Scott oil method, and having no calcium component.

58. The product of claim 57, wherein the calcium concentration is not greater than about 0.2 weight percent, based upon the total weight of the orange juice product.

59. The product of claim 57, wherein the calcium source is a calcium citrate source.

60. The product of claim 57, wherein said low oil condition is one at which terpene in said orange juice product is at a concentration of less than about 60 ppm of the orange juice product.

61. The product of claim 57, wherein said FC orange juice product has substantially no bottom solids.
